(19) United States
(12) Patent Application Publication
(10) Pub. No.: US 2009/0062769 A1
(43) Pub. Date: Mar. 5, 2009

(54) RAPID EXCHANGE CATHETER CONVERTER

(75) Inventors: David M. Graves, Mesa, AZ (US); Sheila Caira, Auburndale, MA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/100,874

(22) Filed: Apr. 10, 2008

Related U.S. Application Data
(60) Provisional application No. 60/911,678, filed on Apr. 13, 2007.

(51) Int. Cl. A61M 25/09

(52) U.S. Cl. 604/510; 600/585; 604/164.13

(57) ABSTRACT

Designs and methods for devices that prevent accidental or inadvertent lateral removal of a guidewire from the guidewire entry tool of a rapid exchange catheter. A first device takes the form of a funnel sized and shaped to slide into the guidewire entry tool. Another illustrative device is shaped and adapted to slide into the guidewire exit slot of the guidewire entry tool, blocking the slot until the converter is removed. In further embodiments, guidewire entry tools include a guidewire exit slot allowing for selective movement of a guidewire laterally from within a catheter. The slot may be designed with structures adapted to prevent inadvertent lateral movement of a guidewire from within the slot. The slot may be formed in a zigzag shape or may include a number of protrusions. Alternatively, the guidewire entry tool may include a split ring that rotates from a first position to a second position, the first position allowing lateral removal of a guidewire and the second position preventing such removal.

### CROSS-REFERENCE TO RELATED APPLICATIONS

This application claims the benefit of U.S. Provisional Ser. No. 60/911,678 filed Apr. 13, 2007, the entire disclosure of which is incorporated herein by reference.

### FIELD OF THE INVENTION

The present invention is related to the field of medical devices. More particularly, the present invention relates to the field of rapid exchange catheters.

### BACKGROUND OF THE INVENTION

Minimally invasive operations using catheters are known for use in a number of different parts of the anatomy and for a number of purposes. Many such procedures are performed with the aid of a guidewire. The guidewire is advanced to a desired anatomical location. A catheter, such as (to name a few illustrative devices) a biliary sphincterotome, a fluid infusion cannula or a balloon catheter, may be advanced over the guidewire to the desired location. In early development of such procedures, the entire catheter would pass over the guidewire, requiring a long guidewire lumen for such an “over the wire” device.

For some procedures, it may become necessary to exchange one catheter for another. With an over-the-wire configuration, exchanging one catheter for another over the guidewire requires a guidewire that is twice as long as the catheter so that the distal end of the guidewire can remain in the desired anatomical location while the catheter is removed. This requires guidewire lengths of several meters, requiring extra hands and hence extra personnel in the operating arena.

To combat the problem of undue guidewire length, among other issues, rapid exchange technologies were developed. These technologies are known as rapid-exchange devices and are designed such that only a distal portion of a catheter tracks over a guidewire. One illustrative rapid exchange technology includes the use of a guidewire channel that is sized to receive a guidewire along the majority of the length of a catheter, with the distal end of the guidewire channel leading into a guidewire lumen. When rapid exchange is desired, the catheter and guidewire are peeled apart, with the guidewire exiting the channel laterally out of the catheter. The channel may take on a U or C cross-section shape, depending on the catheter. Alternatively, the guidewire may be directed into a guidewire lumen that is thinned, slit, perforated, or has an otherwise weakened wall or missing wall portion allowing for lateral removal of the guidewire.

Several rapid exchange devices further include guidewire entry tools that make entry of a guidewire to a guidewire channel easier. FIG. 1 illustrates a prior art guidewire entry tool. The guidewire entry tool 10 includes a proximal end 12 and a distal end 14, with the entry tool 10 tapering from the proximal end to the distal end. The entry tool 10 is attached to a catheter 16. The catheter 16 includes a guidewire lumen that is accessible along a majority of its length, shown as a channel 18. The entry tool 10 directs a guidewire inserted at its proximal end 12 into the guidewire channel 18 of the catheter.

A slot 20 in the entry tool 10 allows a guidewire to be passed out of the entry tool 10 so that the guidewire and catheter 16 may be peeled apart from one another if catheter exchange is desired. In use, it has been noted that on occasion the guidewire can inadvertently slip out of the entry tool 10, making use of the catheter 16 in an over-the-wire manner (i.e. with the guidewire fully inserted into the channel and providing support that improves the pushability and anti-kink characteristics of the catheter 16) difficult, as well as risking loss of the desired anatomical position at the distal end of the catheter 16. Furthermore, if the guidewire does inadvertently move out of the slot 20, it can be difficult to place the guidewire back into its desired location. Finally, the ability of the guidewire to move laterally out of the catheter 16 and entry tool 10 reduces the ability of the guidewire to transmit axial forces, since it is more susceptible to buckling.

### SUMMARY OF THE INVENTION

The present invention, in several illustrative embodiments, includes devices adapted for use with a rapid exchange catheter having a guidewire entry tool. The illustrative devices are converters shaped and sized to couple to a guidewire entry tool and prevent a guidewire passing into a channel/lumen of a catheter through the guidewire entry tool from moving laterally out of the guidewire entry tool. A first illustrative device takes the form of a funnel, sized and shaped to slide into the guidewire entry tool.

Another illustrative converter device is designed for use with a guidewire entry tool having a slot for allowing a guidewire to laterally exit the guidewire entry tool. The converter device is shaped and adapted to slide into the guidewire exit slot of the guidewire entry tool, blocking the slot until the converter is removed. The converter may include a sheath for receiving and passing a guidewire.

In further embodiments, the present invention includes guidewire entry tools including a slot allowing for selective movement of a guidewire laterally from within a catheter. The slot may be designed with structures adapted to prevent inadvertent lateral movement of a guidewire from within the slot. In one such embodiment, the slot is formed in a zigzag shape, while in another embodiment, the guidewire insertion tool includes a number of protrusions that extend partially across the slot.

In yet another embodiment, the guidewire entry tool includes a split ring that can be rotated from a first position to a second position, wherein a guidewire axially passing through the guidewire entry tool is laterally restrained in the first position, and free to move laterally in the second position.

### BRIEF DESCRIPTION OF THE DRAWINGS

FIG. 1 is a perspective view of a prior art guidewire entry tool and catheter;

FIG. 2 is an elevation view from above of a guidewire entry tool and catheter;

FIG. 3 is an elevation view from below of a guidewire blocking member;

FIG. 4 is a side view illustrating how the guidewire blocking member of FIG. 3 engages the guidewire entry tool of FIG. 2;

FIGS. 5A and 5B are cut-away and cross-sectional views of a guidewire blocking insert for use with a guidewire entry tool;

FIGS. 6A and 6B are elevation and cross-sectional views of a guidewire entry tool and catheter;

FIGS. 7A and 7B are cross-sectional and cut-away views of a split ring;

FIGS. 8A-8B are overhead elevation views of a guidewire entry tool incorporating a split ring as in FIGS. 7A-7B;

FIG. 9 is an elevation view of a guidewire entry tool incorporating a number of protrusions in the guidewire exit opening;

FIG. 10 is an elevation view of the guidewire entry tool of FIG. 9 having a guidewire extending therethrough;

FIG. 11 is an elevation view of a guidewire entry tool having a zigzag in the guidewire exit opening;

FIG. 12 is an elevation view of a combination sheath and insert guidewire blocking apparatus;

FIG. 13 is an elevation view of the apparatus of FIG. 12 engaged with a guidewire entry tool; and

FIG. 14 is a perspective view of another illustrative insert guidewire blocking apparatus

### DETAILED DESCRIPTION OF PREFERRED EMBODIMENTS

The following detailed description should be read with reference to the drawings. The drawings, which are not necessarily to scale, depict illustrative embodiments and are not intended to limit the scope of the invention.

Throughout the present description, the term “catheter” should be understood to imply any of various medical devices adapted for insertion to the body, though the term “catheter” need not imply a guidewire. In particular, “catheter” is meant to include cannulae as well as catheters incorporating secondary devices (such as inflatable balloons, cutting wires, or ablation blades) near their distal ends.

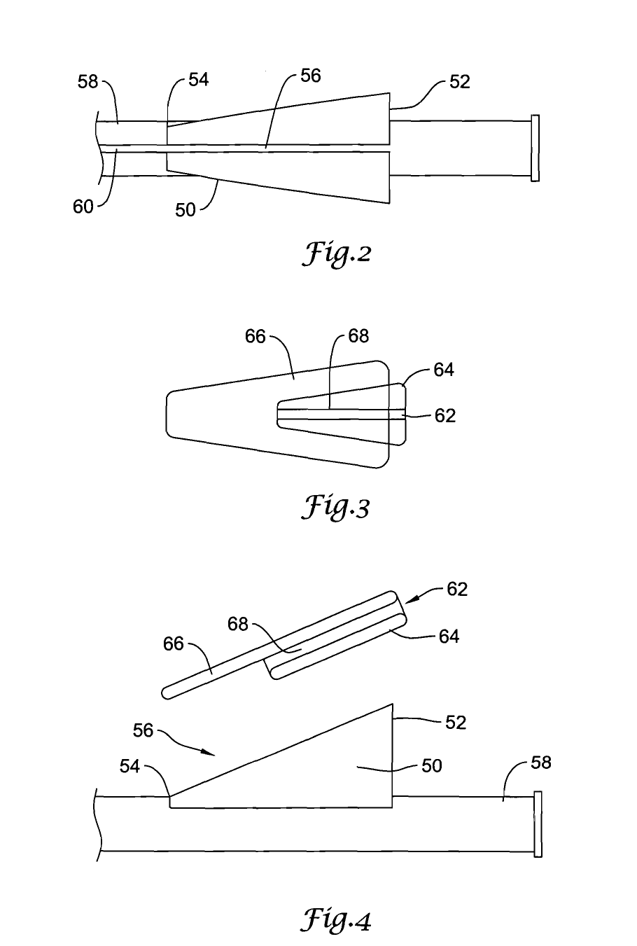

FIG. 2 is an elevation view from above of a guidewire entry tool and catheter. The guidewire entry tool 50 has a proximal end 52 and tapers to a distal end 54, with a guidewire exit opening in the form of a slot 56. The guidewire entry tool 50 is secured to a catheter 58 which includes a guidewire lumen shown in the form of a channel 60. In use, a guidewire is directed into the proximal end 52 of the guidewire entry tool 50 and advanced distally. The taper of the guidewire entry tool 50 directs the guidewire into the guidewire lumen (channel 60) of the catheter 58.

FIG. 3 is an elevation view from below of a guidewire blocking member. The guidewire blocking member 62 includes an inserted portion 64, an external portion 66, and a coupling portion 68 that attaches the inserted portion 64 and external portion 66. The coupling portion 68 is sized and shaped to fit into the slot 56 (FIG. 2) of a corresponding guidewire entry tool 50 (FIG. 2). The external portion 66 is sized and shaped to block off the guidewire exit opening of the guidewire entry tool 50 (FIG. 2), while the inserted portion 64 is sized and shaped to slide into the guidewire entry tool 50 (FIG. 2).

FIG. 4 is a side view illustrating how the guidewire blocking member 62 of FIG. 3 engages the guidewire entry tool 50 of FIG. 2. The guidewire blocking member 62 can be inserted from the proximal end 52 toward the distal end 54 of the guidewire entry tool 50. The coupling piece 68 slides into the groove 56 (noted but not visible), with the insert piece 64 sliding inside the guidewire entry tool 50 and the external piece 66 covering over the groove 56, thereby preventing a guidewire from inadvertently escaping. The guidewire blocking member 62, in a further embodiment, is adapted to be removably coupled to the guidewire entry tool 50 by including a tab or other structure (not shown) allowing a physician to grasp the converter and remove it.

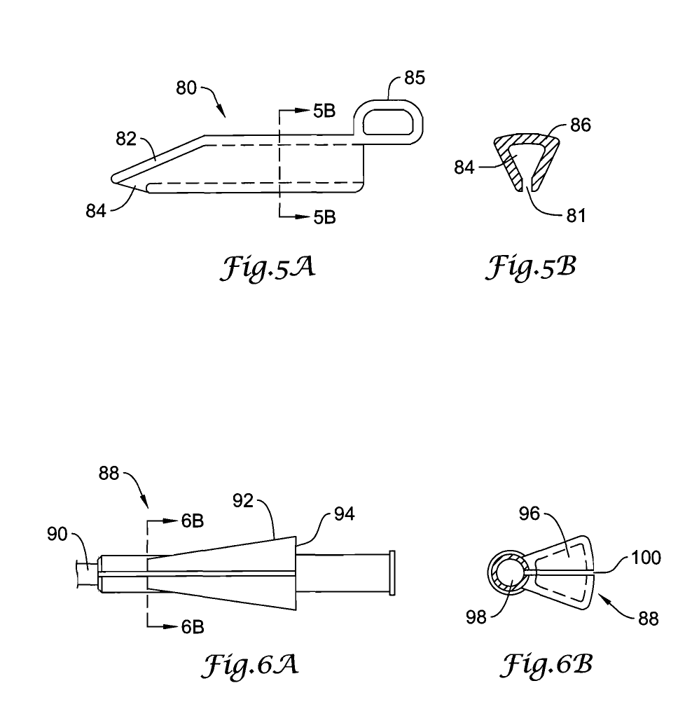

FIGS. 5A and 5B are cut-away and cross-sectional views, respectively, of a guidewire blocking insert for use with a guidewire entry tool. The cross section of FIG. 5B is taken at line 5B-5B of FIG. 5A. As shown in FIG. 5A, the guidewire blocking insert 80 has a tapered end 82 and defines a guidewire lumen. As shown by FIG. 5B, the guidewire lumen 84 is defined by an outer wall 86. A tab, string, or other structure, such as a loop 85, may be attached to the blocking insert 80 to allow for easy removal from a corresponding guidewire entry tool. A longitudinal slit 81 can be included to allow lateral placement and removal of the guidewire blocking insert 80 after sliding longitudinally out of a guidewire entry tool.

FIGS. 6A and 6B are side and cross-sectional views, respectively, of a guidewire entry tool and catheter. It should be noted that the view of FIG. 6B is taken looking proximally from line 6B-6B of FIG. 6A. As shown in the side view of FIG. 6A, the guidewire entry tool 88 is secured to a catheter 90. The guidewire entry tool 88 includes a tapered portion 92 extending distally from its proximal end 94. The cross section of FIG. 6B shows the interior space 96 which is connected to the catheter lumen 98 via a slot 100 that extends up and out of the guidewire entry tool 88. The slot 100 is sized to allow a guidewire to pass laterally therethrough.

Referring generally to FIGS. 5A-5B and 6A-6B, the tapered end 82 of the blocking insert 80 is shaped to slide into the tapered portion 92 of the guidewire entry tool 88. This enables the outer wall 86 of the blocking insert 80 to block off the slot 100, preventing a guidewire entering the catheter 90 through the guidewire entry tool 88 from inadvertently laterally exiting the guidewire entry tool 88.

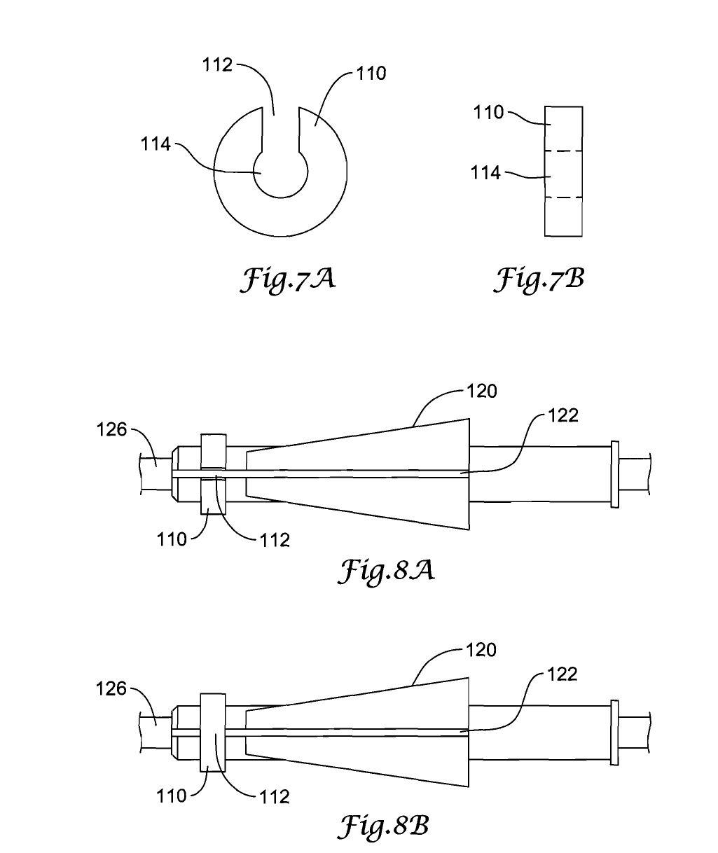

FIGS. 7A and 7B are cross-sectional and cut-away views of a split ring for use in another alternative embodiment. The ring 110 includes a slot 112 that is sized to allow a guidewire to pass therethrough. The ring also defines a lumen 114. The lumen 114 may be sized to rotate around a catheter. The relative size of the slot 112 with respect to the lumen 114 and/or the ring 110 may vary, depending upon the relative guidewire and catheter sizes used in a particular embodiment.

FIGS. 8A-8B are overhead elevation views of a guidewire entry tool incorporating a split ring as in FIGS. 7A-7B. In the illustration of FIG. 8A, a split ring 110 is shown in a first position with the slot 112 in line with a guidewire exit slot for a guidewire entry tool 120 (the guidewire exit slot is shown as a tool slot 122), as well as a channel 126 on the catheter 124. The split ring 110 may also be rotated to the configuration shown in FIG. 8B. In the illustration of FIG. 8B, the slot 112 of the split ring 110 does not align with the tool slot 122 and the channel 126, thereby preventing a guidewire from exiting the guidewire entry tool 120 laterally.

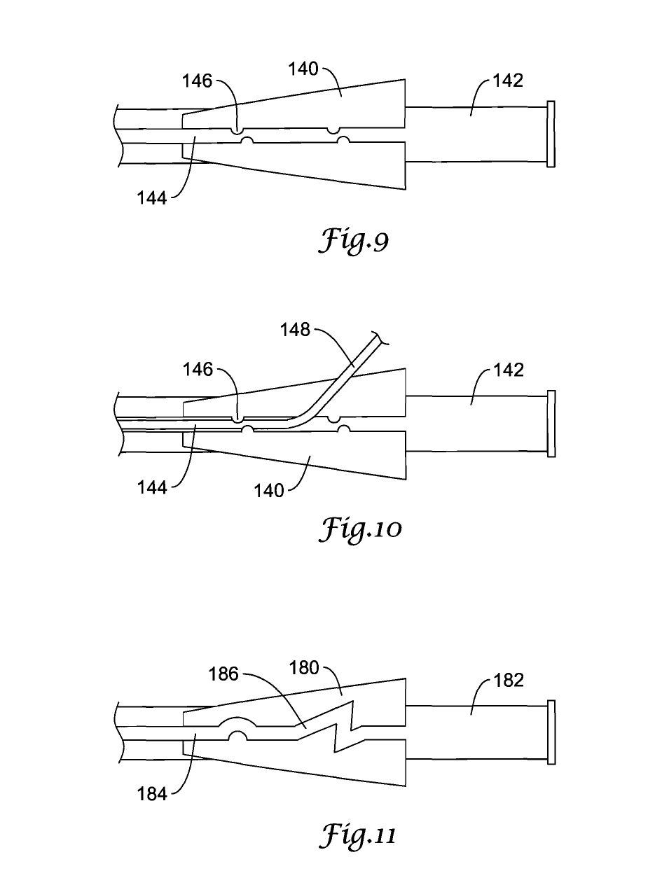

FIG. 9 is an elevation view of a guidewire entry tool incorporating a number of protrusions in the guidewire exit opening. The guidewire entry tool 140 is shown secured to a catheter 142. The guidewire entry tool 140 includes a slot 144. A number of protrusions 146 extend into the slot 144. The protrusions 146 are shown extending about half-way into the slot 144, but do not completely block off the slot 144. Preferably the protrusions 146 leave a gap wide enough to allow a guidewire to pass by the protrusions 146 through the slot 144, but are placed and shaped such that the guidewire must be manipulated intentionally to deform the protrusion and/or get through the gap.

FIG. 10 is an elevation view of the guidewire entry tool of FIG. 9 having a guidewire extending therethrough. The guidewire 148 is shown partially laterally removed from the guidewire entry tool 140. In order to remove the guidewire 148, it must be peeled around the several protrusions 146 to exit the slot 144 laterally. Once laterally removed from the guidewire entry tool 140, the guidewire 148 may then be peeled away from the catheter 142 during a rapid exchange step.

FIG. 11 is an elevation view of a guidewire entry tool having a zigzag in the guidewire exit opening. The guidewire entry tool 180 is shown secured to a catheter 182. The guidewire entry tool 180 includes a guidewire exit opening in the form of a slot 184. The slot 184 defines a path having several changes in direction, including a zigzag 186. Any number of directional changes may be incorporated into the slot 184. In order for a guidewire to exit through the slot 184, the guidewire must be manipulated through the several changes in direction, because ordinarily, the guidewire will not adopt a complicated axial configuration, and will define either a straight line or a single curve.

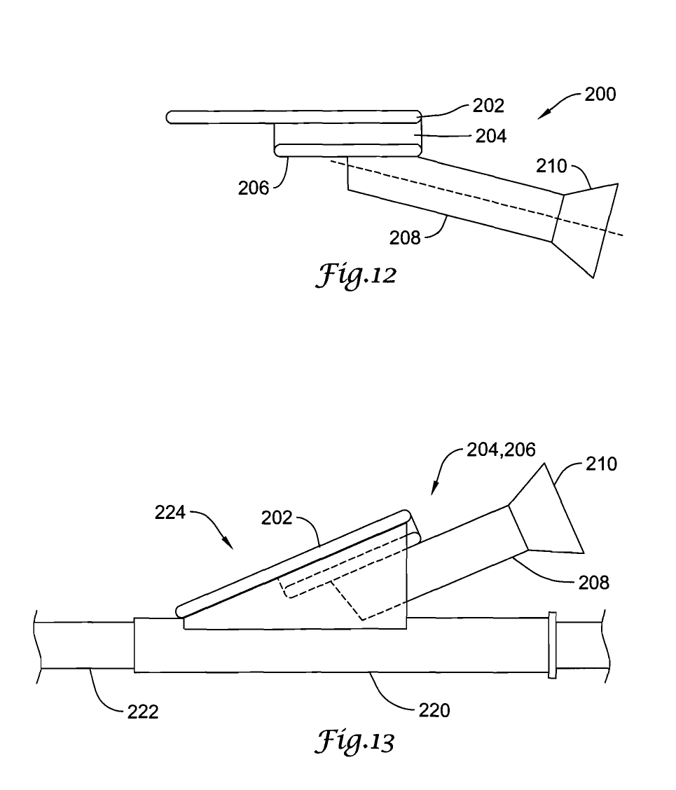

FIG. 12 is an elevation view of a combination sheath and insert guidewire blocking apparatus. The apparatus 200 includes a wing portion 202, an intermediate portion 204, and an insert portion 206, with the insert portion 206 attached to a sheath 208 that includes a flare 210. The flare 210 may be included to make it easier to insert a guidewire at that end. The wing portion 202, intermediate portion 204, and insert portion 206 may be similar to those shown above with respect to FIGS. 3 and 4.

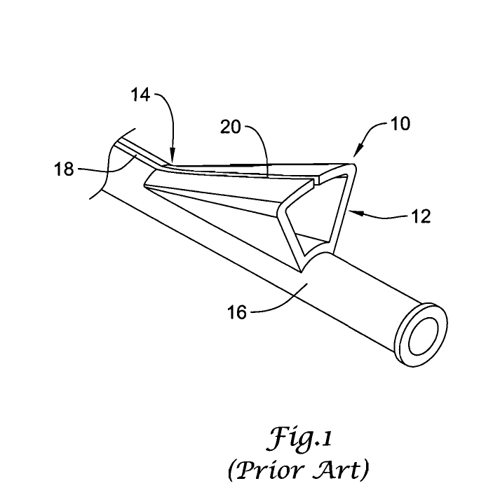

FIG. 13 is an elevation view of the apparatus of FIG. 12 engaged with a guidewire entry tool. The guidewire entry tool 220 is secured to a catheter 222 and includes a guidewire exit opening that takes the form of a channel 224 (not visible, but indicated). The channel 224 may be similar to that shown above in FIG. 1. The intermediate portion 204 passes into the channel 224 such that the wing portion 202 is outside the guidewire entry tool 220, and the insert portion 206 is secured partially inside the guidewire entry tool 220. The sheath 208 enters the guidewire entry tool 220 and prevents a guidewire passing therethrough from exiting the guidewire entry tool 220 via the channel 224. The apparatus 200 can be placed as shown before a guidewire is inserted, with the flare 210 aiding the insertion of a guidewire. Alternatively, the apparatus 200 may pass over a guidewire, with the guidewire passing through the sheath 208, until the intermediate portion 204 enters the channel and removably secures the apparatus 200 to the guidewire entry tool 220.

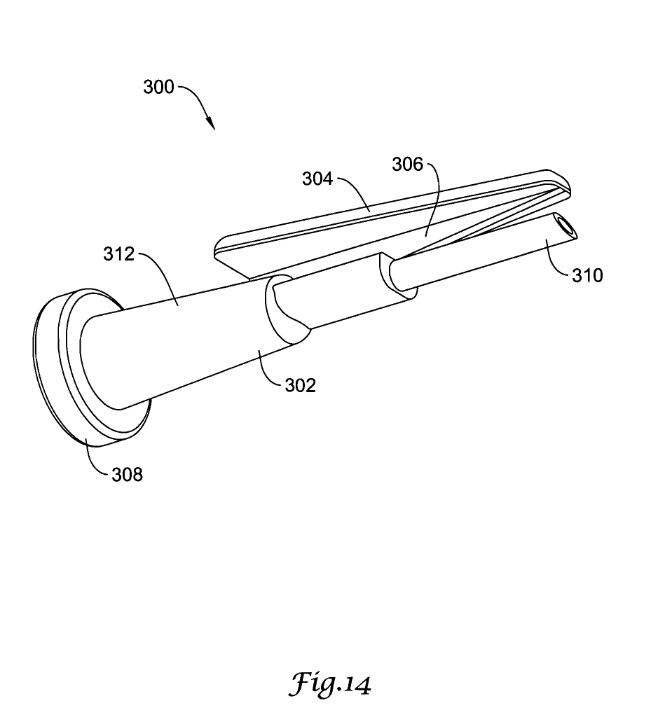

FIG. 14 is a perspective view of another illustrative insert guidewire blocking apparatus. The blocking apparatus 300 includes an insert portion 302, a wing portion 304, and an intermediate portion 306. As with the above embodiments, the apparatus 300 is adapted for use with a guidewire entry tool having a guidewire exit slot into which the intermediate portion 306 can fit. The insert portion 302 includes, for illustrative purposes, a larger proximal end 308 sized to make insertion of a guidewire relatively easy, and tapers to a distal end portion 310. The distal end portion 310 may be sized for insertion to a guidewire channel, and may be relatively flexible and lubricious for allowing easy insertion to a guidewire channel as well as easy insertion of a guidewire therethrough. The insert portion also includes a tapered portion 312 that may be shaped and sized to fit snugly into a guidewire insertion tool. The tapered portion 312 may be sized such that, when inserted fully, the proximal end 308 butts up against the proximal end of a guidewire insertion tool. Alternatively, the guidewire insertion tool, if flexible, may be elastically expanded as the apparatus 300 is inserted such that a friction fit is achieved to keep the apparatus 300 in place. Though a circular shape is shown for the tapered portion 312, other shapes, such as a rounded polygon, may also be provided.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. A device for use with a rapid exchange guidewire entry tool, the guidewire entry tool coupled to a catheter including a guidewire lumen, the catheter having means to allow a guidewire disposed in the guidewire lumen to be laterally removed from the guidewire lumen, the guidewire entry tool adapted to allow axial movement of a guidewire through a slot therein and to allow lateral removal of the guidewire therefrom, the guidewire entry tool disposed to direct a guidewire into the guidewire lumen, the device comprising:
an insert shaped and formed to prevent lateral movement of the guidewire through the slot of the guidewire entry tool when positioned thereon.

2. The device of claim 1, wherein the insert is shaped to conform to a portion of an inside surface of the guidewire entry tool.

3. The device of claim 1, wherein the insert comprises an internal portion, an external portion and a coupling portion connecting the internal portion to the external portion, the coupling portion sized to slide within the slot, the external portion and the internal portion shaped and sized such that, when the coupling portion is slid into a position within the slot, the portion is prevented from moving laterally out through the slot.

4. The device of claim 3, further comprising a sheath attached to the internal portion, the sheath adapted to allow a guidewire to pass therethrough.

5. The device of claim 1, wherein the insert includes a section sized to fit in the guidewire entry tool and a section adapted to allow removal of the device from the guidewire entry tool.

6. A guidewire insertion tool for use as part of a rapid exchange catheter, the catheter including a guidewire lumen having means for allowing a guidewire disposed therein to laterally exit the guidewire lumen, the guidewire insertion tool adapted to direct a guidewire into the guidewire lumen, the guidewire insertion tool comprising:
an exit opening shaped to allow a guidewire directed from outside the catheter through the guidewire insertion tool and into the guidewire lumen to move laterally out of the guidewire insertion tool;
blocking means adapted for impeding lateral movement of the guidewire through the exit opening.

7. The guidewire insertion tool of claim 6, wherein the blocking means includes a protrusion extending across a portion of the exit opening.

8. The guidewire insertion tool of claim 6, wherein the blocking means includes an angled portion of the exit opening.

9. The guidewire insertion tool of claim 6, wherein the blocking means includes a zigzag in the exit opening.

10. The guidewire insertion tool of claim 6, wherein the blocking means includes a moveable piece adapted to move between a first position and a second position, wherein when the moveable piece is in the first position, the exit opening is blocked, and when the moveable piece is in the second position, the exit opening is not blocked.

11. The guidewire insertion tool of claim 10, wherein the moveable piece includes a split ring moveable about a portion of the guidewire insertion tool, the split ring including a gap, wherein the first position arises when the gap is not aligned with the exit opening, and the second position arises when the gap is aligned with the exit opening.

12. The guidewire insertion tool of claim 11, wherein, when the guidewire insertion tool is coupled to the catheter, the split ring is disposed about the catheter.

13. A guidewire insertion tool adapted to direct a guidewire into a guidewire lumen of a rapid exchange catheter, the guidewire insertion tool comprising:
a wall having an exit opening allowing lateral movement of a guidewire out of the guidewire insertion tool, said exit opening being configured to impede lateral movement therethrough.

14. The guidewire insertion tool of claim 13, wherein the wall is shaped such that the exit opening includes a zigzag shape.

15. The guidewire insertion tool of claim 13, wherein the wall includes a number of protrusions extending partially across the exit opening.

16. A method of using a rapid exchange catheter, the rapid exchange catheter including a guidewire lumen having means for allowing lateral removal of a guidewire therefrom, the rapid exchange catheter also including a guidewire insertion tool adapted to direct the guidewire into the guidewire lumen, the guidewire insertion tool defining an exit opening allowing the guidewire to be moved laterally therethrough, the method comprising:
passing a guidewire through the guidewire insertion tool; and
blocking the exit opening.

17. The method of claim 16, wherein the guidewire insertion tool includes a moveable member having a first position wherein the moveable member does not block the exit opening, and a second position wherein the moveable member does block the exit opening, and wherein the step of blocking the exit opening includes moving the movable member from the first position to the second position.

18. The method of claim 16, wherein the step of blocking the exit opening includes passing a blocking member over the guidewire and into the guidewire insertion tool.

19. The method of claim 16, wherein the exit opening comprises a slot, and wherein the step of blocking the exit opening includes sliding a blocking member into the slot.

20. The method of claim 16, further comprising the step of exchanging the catheter by a process including the steps of:
unblocking the exit opening;
with the exit opening unblocked, passing the guidewire laterally out of the exit opening; and
removing the catheter by peeling the catheter away from the guidewire while holding the guidewire in place.
